(19) United States
(12) Patent Application Publication
(10) Pub. No.: US 2008/0319236 A1
(43) Pub. Date: Dec. 25, 2008

(54) CATALYSTS, SYSTEMS AND METHODS FOR ETHER SYNTHESIS

(76) Inventors: Clayton V. McNeff, Andover, MN (US); Larry C. McNeff, Anoka, MN (US); Bingwen Yan, Shoreview, MN (US)

Correspondence Address: PAULY, DEVRIES SMITH & DEFFNER, L.L.C., Plaza VII-Suite 3000, 45 South Seventh Street, MINNEAPOLIS, MN, 55402-1630, US

(21) Appl. No.: 12/146,175

(22) Filed: Jun. 25, 2008

Related U.S. Application Data
(60) Provisional application No. 60/946,093, filed on Jun. 25, 2007.

(51) Int. Cl. C07C 41/09

(52) U.S. Cl. 568/698

(57) ABSTRACT

The present invention relates to methods and catalysts for synthesizing ethers. In an embodiment, the invention includes a process for synthesizing ethers from an alcohol feedstock including heating the alcohol feedstock to a temperature greater than about 100 degrees Celsius; and contacting the alcohol feedstock with a catalyst comprising a metal oxide selected from the group consisting of titania and alumina. Other embodiments are also described herein.

This application claims the benefit of U.S. Provisional Patent Application No. 60/946,093, filed on Jun. 25, 2007, the content of which is herein incorporated by reference in its entirety.

### FIELD OF THE INVENTION

The present invention relates to methods and catalysts for synthesizing ethers. More specifically, the invention relates to methods and catalysts for synthesizing ethers from alcohol feedstocks.

### BACKGROUND OF THE INVENTION

Ethers are chemical compounds of great commercial significance. Ether is the general name for a class of chemical compounds which contain an ether group, an oxygen atom connected to two (substituted) alkyl or aryl groups of general formula R—O—R′. Ethers are commonly used as ingredients in various chemical compositions. They are also commonly used as propellants, solvents and fuels. Ethers can be found in many familiar commercial products from hair spray to cosmetics.

Ethers can be synthesized in various ways. One common technique for the synthesis of symmetric alkyl ethers is the acid-catalyzed condensation of alcohols through nucleophilic substitution. In this reaction, a strong acid (such as sulfuric acid) is added to an alcohol solution and then the reaction mixture is heated. The substitution involves an oxygen nucleophile of one alcohol molecule attacking the electrophilic carbon atom in another alcohol, displacing a water molecule. This reaction is illustrated below:

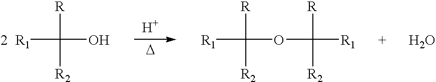

Unfortunately, strong acids are usually highly caustic and can create safety issues. Strong acids can also cause excessive wear on equipment used to carry out the reaction. In addition, recovery or neutralization of the acid after the reaction makes this approach relatively costly and time consuming.

Mixed ethers (unsymmetrical) are frequently produced through a reaction of an alcohol with an alkyl halide and a base yielding an ether and an acid byproduct (such as HCl, HBr, etc.). This reaction is an example of Williamson ether synthesis. Unfortunately, this process also involves reagents and reaction products that can cause excessive wear on equipment, safety issues, and additional processing steps.

Tertiary-butyl ethers are frequently produced by reaction of an alcohol with isobutylene gas in the presence of a strong acid, such as sulfuric acid. Again, however, the use of strong acids can cause excessive wear on equipment, safety issues, and additional processing steps.

For at least these reasons, a need exists for new methods and catalysts for synthesizing ethers from alcohols.

### SUMMARY OF THE INVENTION

The present invention relates to methods and catalysts for synthesizing ethers. In an embodiment, the invention includes a process for synthesizing ethers from an alcohol feedstock including heating the alcohol feedstock to a temperature greater than about 100 degrees Celsius and contacting the alcohol feedstock with a catalyst comprising a metal oxide selected from the group consisting of titania and alumina.

In an embodiment, the invention includes a method of synthesizing ethers including heating an alcohol feedstock to a temperature greater than about 100 degrees Celsius and passing the alcohol feedstock through a housing to form a reaction product mixture. The housing can include a catalyst comprising a metal oxide selected from the group consisting of titania and alumina.

In an embodiment, the invention can include an ether synthesis reactor including a reactor housing, the reactor housing defining an interior volume, a feedstock input port, and a reaction product output port, and a catalyst disposed within the reactor housing, the catalyst comprising a metal oxide selected from the group consisting of titania and alumina.

The above summary of the present invention is not intended to describe each discussed embodiment of the present invention. This is the purpose of the figures and the detailed description that follows.

### BRIEF DESCRIPTION OF THE FIGURES

The invention may be more completely understood in connection with the following drawings, in which:

FIG. 1 is a schematic view of an ether synthesis reactor system in accordance with an embodiment of the invention.

FIG. 2 is a schematic view of an ether synthesis reactor system in accordance with another embodiment of the invention.

FIG. 3 is a schematic view of an ether synthesis reactor system in accordance with another embodiment of the invention.

FIG. 4 is a schematic view of an ether synthesis reactor system in accordance with another embodiment of the invention.

FIG. 5 is a graph of an NIR spectrum of a product gas according to example 3 below.

FIG. 6 is a graph of an NIR spectrum of a product gas according to example 4 below.

FIG. 7 is a graph of an NMR spectrum of a product liquid according to example 4 below.

FIG. 8 is a graph of an NIR spectrum of a product gas according to example 5 below.

FIG. 9 is a graph of an NIR spectrum of a product gas according to example 5 below.

FIG. 10 is a graph of an NMR spectrum of a product liquid according to example 5 below.

FIG. 11 is a graph of an NMR spectrum of a product liquid according to example 5 below.

While the invention is susceptible to various modifications and alternative forms, specifics thereof have been shown by way of example and drawings, and will be described in detail. It should be understood, however, that the invention is not limited to the particular embodiments described. On the contrary, the intention is to cover modifications, equivalents, and alternatives falling within the spirit and scope of the invention.

### DETAILED DESCRIPTION OF THE INVENTION

The embodiments of the present invention described herein are not intended to be exhaustive or to limit the invention to the precise forms disclosed in the following detailed description. Rather, the embodiments are chosen and described so that others skilled in the art can appreciate and understand the principles and practices of the present invention.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

As described above, ethers are commercially valuable chemical compounds with many different commercial applications. Unfortunately, current techniques for synthesizing ethers involve the use of strong acids which are dangerous to handle, cause significant wear on equipment, and must be removed from the final product creating additional and expensive processing steps.

However, as demonstrated herein, the synthesis of ethers from alcohols can be efficiently catalyzed by certain metal oxides. In an embodiment, the invention includes a process for producing ethers from an alcohol feedstock including the operations of heating the alcohol feedstock to a temperature greater than about 150 degrees Celsius and passing the alcohol feedstock over a catalyst comprising a metal oxide selected from the group consisting of zirconia, hafnia, titania and alumina. In some embodiments, the metal oxide is selected from the group consisting of titania and alumina.

While not intending to be bound by theory, it is believed that the use of metal oxides to catalyze the synthesis of ethers can offer various advantages. For example, metal oxide catalysts used with embodiments of the invention are extremely durable making them conducive to use in many different potential processing steps. In addition, such metal oxide catalysts can be reused many times, making this approach cost effective. Further, metal oxide catalysts used with embodiments of the invention do not create the same types of handling hazards created by the use of caustic acids, such as sulfuric acid.

Metal oxide catalysts used with embodiments of the invention can include metal oxides with surfaces including Lewis acid sites, Bronsted base sites, and Bronsted acid sites. By definition, a Lewis acid is an electron pair acceptor. A Bronsted base is a proton acceptor and a Bronsted acid is a proton donor. Metal oxide catalysts of the invention can specifically include zirconia, alumina, titania and hafnia. Metal oxide catalysts of the invention can also include silica clad with a metal oxide selected from the group consisting of zirconia, alumina, titania, hafnia, zinc oxide, copper oxide, magnesium oxide and iron oxide. In some embodiments, the metal oxide catalyst can be of a single metal oxide type. By way of example, in some embodiments, the metal oxide catalyst is substantially pure titania. In some embodiments, the metal oxide catalyst is substantially pure alumina. Metal oxide catalysts of the invention can also include mixtures of metal oxides, such as mixtures of metal oxides including zirconia, alumina, titania and/or hafnia. Of the various metal oxides that can be used with embodiments of the invention, zirconia, titania, alumina and hafnia are advantageous as they are very chemically and thermally stable and can withstand very high temperatures and pressures as well as extremes in pH. Titania and alumina are advantageous because of the additional reason that they are less expensive materials.

Metal oxides of the invention can include metal oxide particles clad with carbon. Carbon clad metal oxide particles can be made using various techniques such as the procedures described in U.S. Pat. Nos. 5,108,597; 5,254,262; 5,346,619; 5,271,833; and 5,182,016, the contents of which are herein incorporated by reference. Carbon cladding on metal oxide particles can render the surface of the particles more hydrophobic.

Metal oxides of the invention can also include polymer coated metal oxides. By way of example, metal oxides of the invention can include a metal oxide coated with polybutadiene (PBD). Polymer coated metal oxide particles can be made using various techniques such as the procedure described in Example 1 of U.S. Pub. Pat. App. No. 2005/0118409, the contents of which are herein incorporated by reference. Polymer coatings on metal oxide particles can render the surface of the particles more hydrophobic.

Metal oxide catalysts of the invention can be made in various ways. As one example, a colloidal dispersion of zirconium dioxide can be spray dried to produce aggregated zirconium dioxide particles. Colloidal dispersions of zirconium dioxide are commercially available from Nyacol Nano Technologies, Inc., Ashland, Mass. The average diameter of particles produced using a spray drying technique can be varied by changing the spray drying conditions. Examples of spray drying techniques are described in U.S. Pat. No. 4,138,336 and U.S. Pat. No. 5,108,597, the contents of both of which are herein incorporated by reference. It will be appreciated that other methods can also be used to create metal oxide particles. One example is an oil emulsion technique as described in Robichaud et al., Technical Note, “An Improved Oil Emulsion Synthesis Method for Large, Porous Zirconia Particles for Packed- or Fluidized-Bed Protein Chromatography,” Sep. Sci. Technol. 32, 2547-59 (1997). A second example is the formation of metal oxide particles by polymer induced colloidal aggregation as described in M. J. Annen, R. Kizhappali, P. W. Carr, and A. McCormick, “Development of Porous Zirconia Spheres by Polymerization-Induced Colloid Aggregation-Effect of Polymerization Rate,” J. Mater. Sci. 29, 6123-30 (1994). A polymer induced colloidal aggregation technique is also described in U.S. Pat. No. 5,540,834, the contents of which are herein incorporated by reference.

Metal oxide catalysts used in embodiments of the invention can be sintered by heating them in a furnace or other heating device at a relatively high temperature. In some embodiments, the metal oxide is sintered at a temperature of about 160° C. or greater. In some embodiments, the metal oxide is sintered at a temperature of about 400° C. or greater. In some embodiments, the metal oxide is sintered at a temperature of about 600° C. or greater. Sintering can be done for various amounts of time depending on the desired effect. Sintering can make metal oxide catalysts more durable. In some embodiments, the metal oxide is sintered for more than about 30 minutes. In some embodiments, the metal oxide is sintered for more than about 3 hours. However, sintering also reduces the surface area. In some embodiments, the metal oxide is sintered for less than about 1 week.

In some embodiments, the metal oxide catalyst is in the form of particles. Particles within a desired size range can be specifically selected for use as a catalyst. For example, particles can be sorted by size using techniques such as air classification, elutriation, settling fractionation, or mechanical screening. In some embodiments, the size of the particles is greater than about 0.2 μm. In some embodiments, the size range selected is from about 0.2 μm to about 1 mm. In some embodiments, the size range selected is from about 1 μm to about 100 μm. In some embodiments, the size range selected is from about 5 μm to about 15 μm. In some embodiments, the average size selected is about 10 μm. In some embodiments, the average size selected is about 5 μm.

In some embodiments, metal oxide particles used with embodiments of the invention are porous. By way of example, in some embodiments the metal oxide particles can have an average pore size of about 30 angstroms to about 2000 angstroms. However, in other embodiments, metal oxide particles used are non-porous.

The physical properties of a porous metal oxide can be quantitatively described in various ways such as by surface area, pore volume, porosity, and pore diameter. In some embodiments, metal oxide catalysts of the invention can have a surface area of between about 1 and about 200 m²/gram. Pore volume refers to the proportion of the total volume taken up by pores in a material per weight amount of the material. In some embodiments, metal oxide catalysts of the invention can have a pore volume of between about 0.01 mL/g and about 2 mL/g. Porosity refers to the proportion within a total volume that is taken up by pores. As such, if the total volume of a particle is 1 cm³ and it has a porosity of 0.5, then the volume taken up by pores within the total volume is 0.5 cm³. In some embodiments, metal oxide catalysts of the invention can have a porosity of between about 0 and about 0.8. In some embodiments, metal oxide catalysts of the invention can have a porosity of between about 0.3 and 0.6.

Metal oxide particles used with embodiments of the invention can have various shapes. By way of example, in some embodiments the metal oxide can be in the form of spherules. In other embodiments, the metal oxide can be a monolith. In some embodiments, the metal oxide can have an irregular shape.

The Lewis acid sites on metal oxides of the invention can interact with Lewis basic compounds. Thus, in some embodiments, Lewis basic compounds can be bonded to the surface of metal oxides. However, in other embodiments, the metal oxides used with embodiments herein are unmodified and have no Lewis basic compounds bonded thereto. A Lewis base is an electron pair donor. Lewis basic compounds of the invention can include anions formed from the dissociation of acids such as hydrobromic acid, hydrochloric acid, hydroiodic acid, nitric acid, sulfuric acid, perchloric acid, boric acid, chloric acid, phosphoric acid, pyrophosphoric acid, chromic acid, permanganic acid, phytic acid and ethylenediamine tetramethyl phosphonic acid (EDTPA), and the like. Lewis basic compounds of the invention can also include hydroxide ion as formed from the dissociation of bases such as sodium hydroxide, potassium hydroxide, lithium hydroxide and the like.

The anion of an acid can be bonded to a metal oxide of the invention by refluxing the metal oxide in an acid solution. By way of example, metal oxide particles can be refluxed in a solution of sulfuric acid. Alternatively, the anion formed from dissociation of a base, such as the hydroxide ion formed from dissociation of sodium hydroxide, can be bonded to a metal oxide by refluxing in a base solution. By way of example, metal oxide particles can be refluxed in a solution of sodium hydroxide. The base or acid modification can be achieved under exposure to the acid or base in either batch or continuous flow conditions when disposed in a reactor housing at elevated temperature and pressure to speed up the adsorption/modification process. In some embodiments, fluoride ion, such as formed by the dissociation of sodium fluoride, can be bonded to the particles.

In some embodiments, metal oxide particles can be packed into a housing, such as a column. Disposing metal oxide particles in a housing is one approach to facilitating continuous flow processes. Many different techniques may be used for packing the metal oxide particles into a housing. The specific technique used may depend on factors such as the average particle size, the type of housing used, etc. Generally speaking, particles with an average size of about 1-20 microns can be packed under pressure and particles with an average size larger than 20 microns can be packed by dry-packing/tapping methods or by low pressure slurry packing. In some embodiments, the metal oxide particles of the invention can be impregnated into a membrane, such as a PTFE membrane.

However, in some embodiments, metal oxide catalysts used with embodiments of the invention are not in particulate form. For example, a layer of a metal oxide can be disposed on a substrate in order to form a catalyst used with embodiments of the invention. The substrate can be a surface that is configured to contact the alcohol feedstock during processing. In one approach, a metal oxide catalyst can be disposed as a layer over a surface of a reactor that contacts the alcohol feedstock. Alternatively, the metal oxide catalyst can be embedded as a particulate in the surface of an element that is configured to contact the alcohol feedstock during processing.

In some embodiments, an additive can be added to the alcohol feedstock before or during processing. Additives can include water, carrier compounds, and the like.

It is believed that the synthesis of ethers from alcohol feedstocks (etherification) using a metal oxide catalyst is temperature dependent. If the temperature is not high enough, the synthesis reaction will not proceed optimally. As such, in some embodiments, the alcohol feedstock is heated to about 150° Celsius or hotter. In some embodiments, the alcohol feedstock is heated to about 200° Celsius or higher. In some embodiments, the alcohol feedstock is heated to about 300° Celsius or higher. In some embodiments, the alcohol feedstock is heated to a temperature of between about 150° Celsius and about 400° Celsius. In some embodiments, the alcohol feedstock is heated to a temperature of between about 180° Celsius and about 220° Celsius. In some embodiments, the temperature is greater than the critical temperature for the alcohol used.

In an embodiment, the contact time is between about 0.1 seconds and 2 hours. In an embodiment, the contact time is between about 1 second and 20 minutes. In an embodiment, the contact time is between about 2 seconds and 1 minute.

### Ether Synthesis Reactors

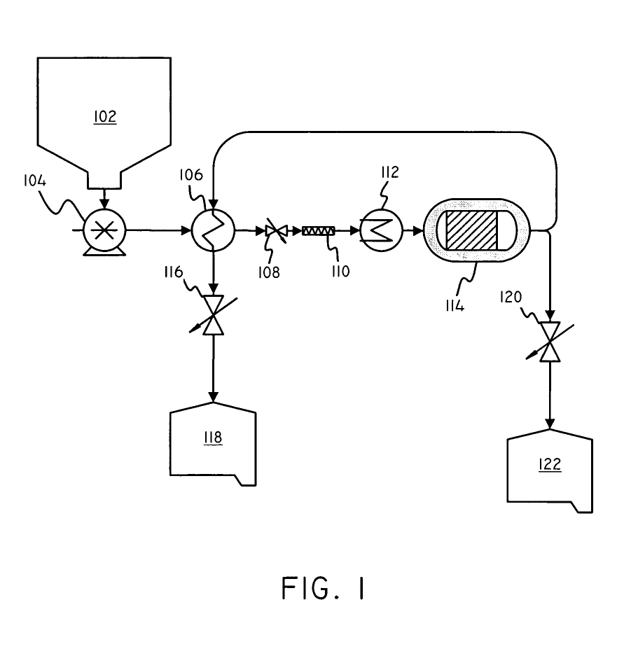

It will be appreciated that many different reactor designs are possible in order to perform methods and processes as described herein. Specific design choices can be influenced by various factors including, significantly, the nature of the alcohol feedstock. Referring now to FIG. 1, a schematic diagram is shown of an ether synthesis reactor in accordance with an embodiment of the invention. In this embodiment, an alcohol feedstock is held in an alcohol feedstock tank 102. In some embodiments, the alcohol feedstock tank 102 can be heated.

The alcohol feedstock then passes through a pump 104 before passing through a heat exchanger 106 where the feedstock absorbs heat from downstream products. An exemplary counter-flow heat exchanger is described in U.S. Pat. No. 6,666,074, the contents of which are herein incorporated by reference. For example, a pipe or tube containing the effluent flow is routed past a pipe or tube holding the feedstock flow or the reaction mixture. In some embodiments, a thermally conductive material, such as a metal, connects the effluent flow with the feedstock flow so that heat can be efficiently transferred from the effluent products to the incoming feedstock. Transferring heat from the effluent flow to the feedstock flow can make the production process more energy efficient since less energy is used to get the reaction mixture up to the desired temperature.

The alcohol feedstock can be continuously sparged with an inert gas such as nitrogen to remove dissolved oxygen from the feedstock. The alcohol feedstock passes through a shutoff valve 108 and, optionally, a filter 110 to remove particulate material of a certain size from the feedstock stream. The alcohol feedstock then passes through a preheater 112. The preheater 112 can elevate the temperature of the reaction mixture to a desired level. Many different types of heaters are known in the art and can be used.

The reaction mixture can then pass through a reactor 114 where the alcohol feedstock is converted into a reaction product mixture including ethers. The reactor can include a metal oxide catalyst, such as in the various forms described herein. In some embodiments the reactor housing is a ceramic that can withstand elevated temperatures and pressures. In some embodiments, the reactor housing is a metal or an alloy of metals.

Depending on the particular alcohols in the feedstock, newly synthesized ethers can include both gases and liquids. Gases coming off the reactor, such as volatile ethers, can pass through a backpressure regulator 120 before being collected in a gas product collection tank 122. The rest of the reaction product mixture can pass through the heat exchanger 106 in order to transfer heat from the effluent reaction product stream to the alcohol feedstock stream. The liquid reaction product mixture can also pass through a backpressure regulator 116 before passing on to a liquid reaction product storage tank 118. In some embodiments, residual alcohol can be separated from the liquid reaction product mixture and then fed back into the reactor or back into the alcohol feedstock tank 102.

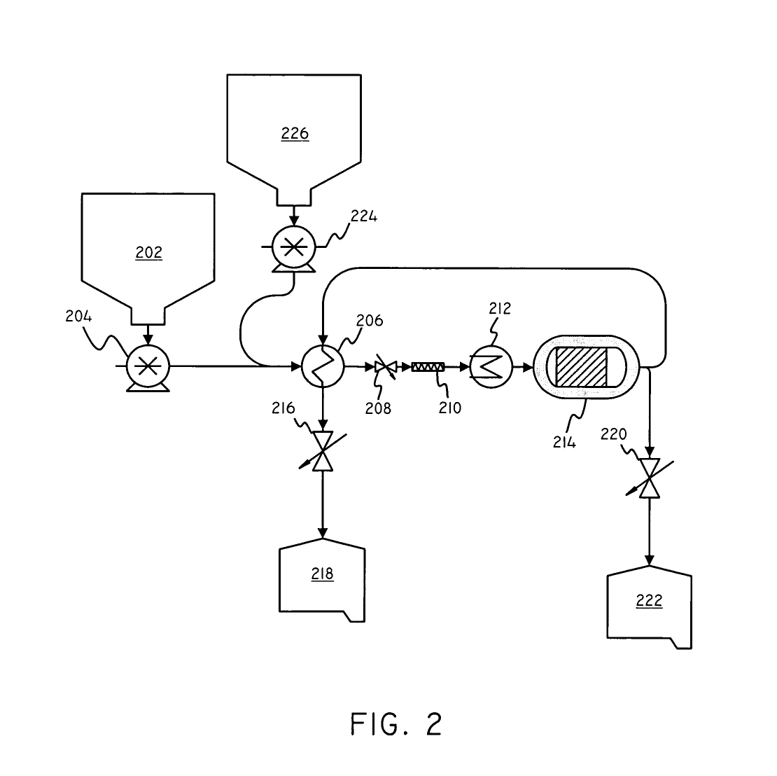

In some embodiments, a plurality of different alcohol feedstocks can be used to form ethers. For example, where a mixed ether is to be formed, a first alcohol can be supplied from a first alcohol feedstock tank and a second alcohol can be supplied from a second alcohol feedstock tank. Referring now to FIG. 2, a schematic view of an ether synthesis reactor is presented in accordance with another embodiment of the invention. In this embodiment, a first alcohol feedstock is held in a first alcohol feedstock tank 202. A second alcohol can be held in a second alcohol feedstock tank 226. In some embodiments, one or both of the first and second alcohol feedstock tanks can be heated. The alcohol feedstock tank may be continuously sparged with an inert gas such as nitrogen to remove dissolved oxygen from the feedstock.

The alcohol feedstocks then pass from the first alcohol feedstock tank 202 and second alcohol feedstock tank 226 through pumps 204 and 224, respectively, before being combined and passing through a heat exchanger 206 where the feedstocks absorb heat from downstream products. The alcohol feedstock mixture then passes through a shutoff valve 208 and, optionally, a filter 210. The alcohol feedstock mixture then passes through a preheater 212 and through a reactor 214 where the alcohol feedstock is converted into a reaction product mixture including ethers. The reactor can include a metal oxide catalyst, such as in the various forms described herein.

Depending on the particular alcohols in the feedstock, newly synthesized ethers can include both gases and liquids. Gases coming off the reactor, such as volatile ethers, can pass through a backpressure regulator 220 before being collected in a gas product collection tank 222. The rest of the reaction product mixture can pass through the heat exchanger 206 in order to transfer heat from the effluent reaction product stream to the alcohol feedstock stream. The liquid reaction product mixture can also pass through a backpressure regulator 216 before passing on to a liquid reaction product storage tank 218. In some embodiments, the alcohol feedstock can be completely converted to an ether product. In other embodiments, a portion of the alcohol feedstock is converted to an ether product and there is an amount of residual alcohol leftover. In some embodiments, residual alcohol can be separated from the liquid reaction product mixture and then fed back into the reactor or back into the alcohol feedstock tanks.

In some embodiments, the alcohol feedstock is kept under pressure during the reaction in order to prevent components of the reaction mixture (the alcohol feedstock and any additives) from vaporizing. The reactor housing can be configured to withstand the pressure under which the reaction mixture is kept. In addition, a backpressure regulator can be used to maintain a desired pressure. A desirable pressure for the reactor can be estimated with the aid of the Clausius-Clapeyron equation. Specifically, the Clausius-Clapeyron equation can be used to estimate the vapor pressures of a liquid. The Clausius-Clapeyron equation is as follows:

$$ln ( \frac{{P}_{1}}{{P}_{2}} ) = \frac{Δ {H}_{vap}}{R} ( \frac{1}{{T}_{2}} - \frac{1}{{T}_{1}} )$$

wherein ΔHᵥₐₚ is the enthalpy of vaporization; P₁ is the vapor pressure of a liquid at temperature T₁; P₂ is the vapor pressure of a liquid at temperature T₂, and R is the ideal gas constant.

In an embodiment, the pressure inside the housing is greater than the vapor pressures of any of the components of the reaction mixture. In an embodiment, the pressure is greater than about 500 psi. In an embodiment, the pressure is greater than about 800 psi. In an embodiment, the pressure is greater than about 1000 psi. In an embodiment, the pressure is greater than about 1500 psi. In an embodiment, the pressure is greater than about 2000 psi. In an embodiment, the pressure is greater than about 3000 psi. In an embodiment, the pressure is greater than about 3000 psi. In an embodiment, the pressure is greater than about 4000 psi. In an embodiment, the pressure is greater than about 5000 psi. In some embodiments, the pressure is greater than the critical pressure for the alcohol being used.

The reaction mixture may be passed over the metal oxide catalyst for a length of time sufficient for the reaction to reach a desired level of completion. This will, in turn, depend on various factors including the temperature of the reaction, the chemical nature of the catalyst, the surface area of the catalyst, the contact time with the catalyst and the like.

In some embodiments, the reaction mixture reaches the desired level of completion after one pass over the metal oxide catalyst bed or packing. However, in some embodiments, the effluent flow may be rerouted over the same metal oxide catalyst or routed over another metal oxide catalyst bed or packing so that reaction is pushed farther toward completion in stages.

In some embodiments two or more metal oxide catalyst beds can be used to convert alcohol feedstocks to ether products. In some embodiments, an acid-modified metal oxide catalyst (such as sulfuric or phosphoric acid modified) and a base-modified metal oxide catalyst (such as sodium hydroxide modified) can be separately formed but then disposed together within a single reactor housing. In such an approach, the reaction mixture passing through the reactor housing can be simultaneously exposed to both the acid and base modified metal oxide catalysts.

In some embodiments, two different metal oxides (such zirconia and titania) can be separately formed but then disposed together within a single reactor housing. In such an approach, the reaction mixture passing through the reactor housing can be simultaneously exposed to both metal oxide catalysts.

In some embodiments, one or more metal oxides (such as zirconia and titania) can be coated on an inert porous support (such as silica gel or zeolite) separately formed but then disposed together within a single reactor housing. In such an approach, the reaction mixture passing through the reactor housing can be simultaneously exposed to the metal oxide catalyst(s).

### Alcohol Feedstocks

It will be appreciated that many different alcohols can be used herein in order to synthesize ethers. Exemplary alcohols can include aliphatic, aromatic, and alicyclic alcohols. In some embodiments, alcohols can include C1-C30 alcohols (alcohols with one to thirty carbon atoms). In some embodiments, alcohols can include C1-C6 alkyl alcohols. Alcohols used herein can be mono-functional or multi-functional (e.g., one alcohol moiety or multiple alcohol moieties). Exemplary alcohols can specifically include methanol, ethanol, propanol, isopropyl alcohol, butanol, and the like.

Alcohol feedstocks used with embodiments herein can include those formed through fermentation processes. By way of example, biomass can be fermented by microorganisms in order to produced alcohol feedstocks. Virtually any living organism is a potential source of biomass for use in fermentation processes. As such, alcohol feedstocks can be derived from industrial processing wastes, food processing wastes, mill wastes, municipal/urban wastes, forestry products and forestry wastes, agricultural products and agricultural wastes, amongst other sources.

Though not limiting the scope of possible sources, specific examples of biomass crop sources for alcohol production can include corn, poplar, switchgrass, reed canary grass, willow, silver maple, black locust, sycamore, sweetgum, sorghum, miscanthus, eucalyptus, hemp, maize, wheat, soybeans, alfalfa, and prairie grasses.

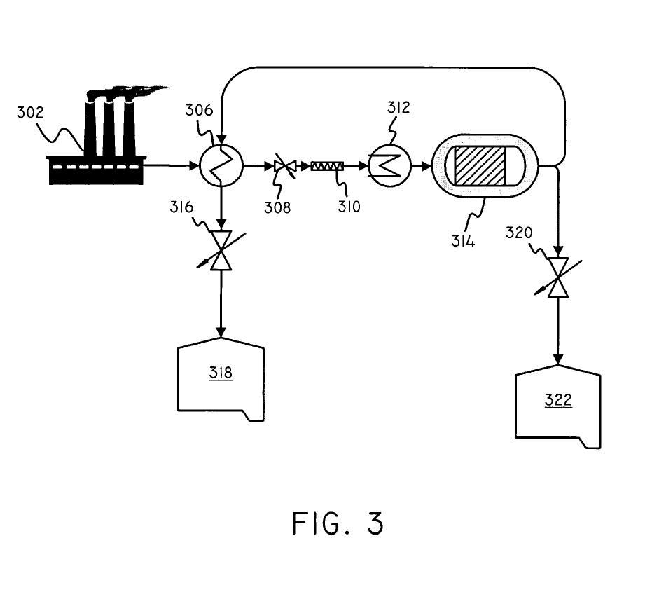

In some embodiments, ether synthesis reactors as described herein can be used in conjunction with fermentation plants that produce suitable alcohol feedstocks. For example, referring now to FIG. 3, a schematic diagram is shown of an ether synthesis plant in accordance with an embodiment of the invention. In this embodiment, an alcohol feedstock is produced by an alcohol production plant 302. The alcohol production plant 302 can include various components including stirred tank reactors, distillation equipment, and the like. Exemplary components and microorganisms for an alcohol production plant are described in U.S. Pat. Nos. 4,425,433; 4,808,526; 5,231,017; and 7,135,308, the contents of which are herein incorporated by reference. The alcohol feedstock then passes through a heat exchanger 306 where the feedstock absorbs heat from downstream products.

The alcohol feedstock may be continuously sparged with an inert gas such as nitrogen to remove dissolved oxygen from the feedstock. The alcohol feedstock passes through a shutoff valve 308 and, optionally, a filter 310 to remove particulate material of a certain size from the feedstock stream. The alcohol feedstock then passes through a preheater 312. The reaction mixture can then pass through a reactor 314 where the alcohol feedstock is converted into a reaction product mixture including ethers. The reactor can include a metal oxide catalyst, such as in the various forms described herein.

Depending on the particular alcohols in the feedstock, newly synthesized ethers can include both gases and liquids. Gases coming off the reactor, such as volatile ethers, can pass through a backpressure regulator 320 before being collected in a gas product collection tank 322. The rest of the reaction product mixture can pass through the heat exchanger 306 in order to transfer heat from the effluent reaction product stream to the alcohol feedstock stream. The liquid reaction product mixture can also pass through a backpressure regulator 316 before passing on to a liquid reaction product storage tank 318. In some embodiments, residual alcohol can be separated from the liquid reaction product mixture and then fed back into the reactor.

The present invention may be better understood with reference to the following examples. These examples are intended to be representative of specific embodiments of the invention, and are not intended as limiting the scope of the invention.

### EXAMPLES

### Example 1

### Formation of Base Modified Titania Particles

700 mL of 1.0 M sodium hydroxide was placed in a 2 liter plastic Erlenmeyer flask. 110 g of 80 μm diameter (60 Angstrom average pore diameter) bare titania (commercially available from ZirChrom Separations, Inc., Anoka, Minn.) was added to the flask. The particle suspension was sonicated for 10 minutes under vacuum and then swirled for 2 hours at ambient temperature. The particles were then allowed to settle and the alkaline solution was decanted and then 1.4 liters of HPLC-grade water was added to the flask followed by settling and decanting. Then 200 mL of HPLC-grade water was added to the flask and the particles were collected on a Millipore nylon filter with 0.45 micron pores. The collected particles were then washed with 2 aliquots of 200 mL HPLC-grade water followed by 3 aliquots of 200 mL of HPLC-grade methanol. Air was then allowed to pass through the particles until they were free-flowing.

### Example 2

### Formation of a Packed Column

Particles as formed in Example 1 were dry packed into two 10.0 mm i.d.×15 cm stainless steel reactor tubes. Each tube contained 16.3 g of the base modified titania.

### Example 3

### Synthesis of Ether from Methanol

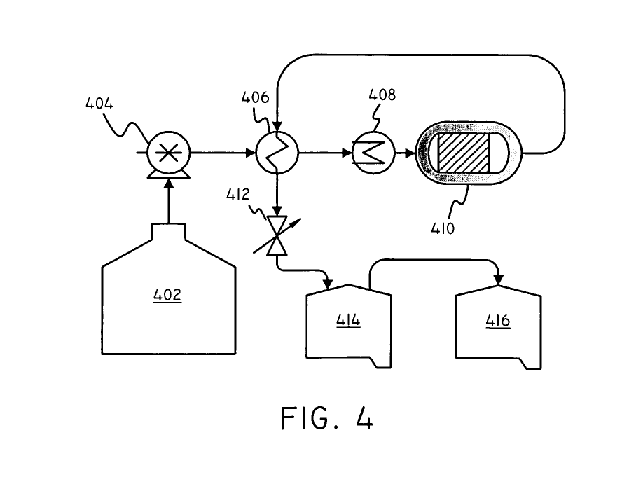

A reactor system was set up as shown in FIG. 4. The system included one high-pressure pump 404 (Waters 590 programmable pump) connected to a methanol reservoir 402 that was continuously sparged with nitrogen to remove dissolved oxygen from the methanol feedstock. The methanol was then pumped into a heat exchanger 406 where heat from the hot from the fixed bed catalytic reactor 410 was exchanged with the incoming stream of methanol. The fixed bed catalytic reactor 410 was a 10 mm i.d.×15 cm column packed with base modified titania, prepared as described in example 2 above. After the heat exchanger 406, the methanol passed through a preheater 408 capable of bringing the mixture to the desired set point temperature before it entered the fixed bed catalytic reactor 410. The reactor 410 included an independent thermostat. The backpressure of the system was maintained through the use of a backpressure regulator 412.

After passing through the heat exchanger 406, the “cooled” product mixture was collected into a vacuum flask 414 attached to a “cold finger” (acetone/ice mixture bath) to continuously condense the less volatile components of the vapor into a liquid state. The gas that was evolved during the reaction that does not get trapped by the “cold finger” was collected into a gas collection flask 416 using a water displacement method.

The reaction conditions for this example are summarized below in Table 1. Notably, the reaction was tested at two different methanol flow rates, 7.00 g/min and 3.52 g/min.

TABLE 1

| | | Reactor | | Outlet Temp. | | | MeOH | Gases |
|---|---|---|---|---|---|---|---|---|
| Preheater | Reactor | Outlet | Inlet Temp. of | of heater | Front | Back | Flow | collection |
| Temp. | Inlet | Temp. | heater | exchanger | Pressure | Pressure | Rate | Rate |
| (° C.) | Temp. (° C.) | (° C.) | exchanger (° C.) | (° C.) | (PSI) | (PSI) | (g/min) | (L/min) |
| 350 | 350 | 351 | 273 | 168 | 2500 | 2500 | 3.522 | 0.162 |
| 350 | 350 | 351 | 284 | 165 | 2500 | 2500 | 7.000 | 0.129 |

The resulting gas was shown to be combustible by igniting the same and observing it to burn. The mass balance (liquid in versus liquid out) of chemical reactions is shown below in Table 2.

TABLE 2

| MeOH | MeOH | | | | |
|---|---|---|---|---|---|
| Flow | Flow | Cold Well | MeOH | | MeOH to |
| (g/min) | (g/min) at | Collection | Conversion | MeOH | Gases |
| at Inlet | Outlet | (g/min) | rate (g/min) | Recovery | Conversion |
| 3.522 | 3.100 | 0.025 | 0.398 | 88.7% | 11.3% |
| 7.000 | 6.500 | 0.040 | 0.460 | 93.4% | 6.6% |

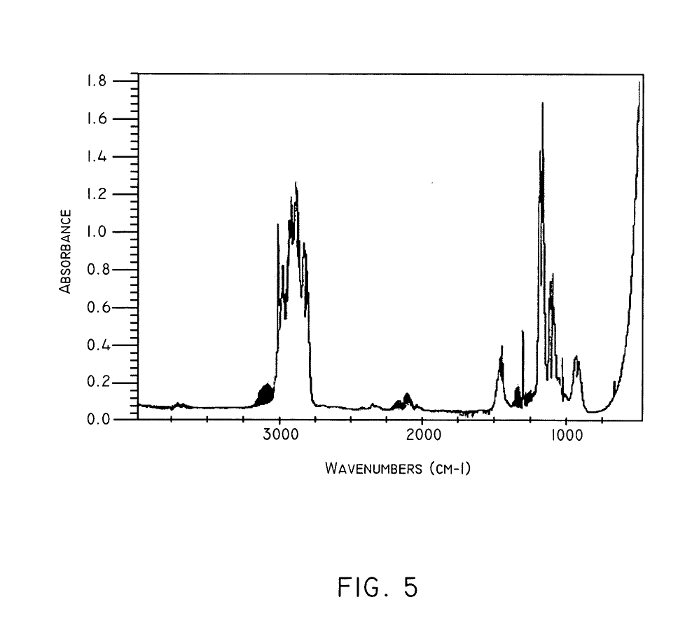

A sample of gas from the reactor was also collected in an IR cell for NIR (Near Infra-Red) analysis. The NIR spectrum is shown in FIG. 5 for the gas produced with a methanol flow rate of 3.52 g/min. The NIR spectrum for the gas was compared with standard curve NIR spectra for pure dimethyl ether, methane, and carbon dioxide. NIR results indicate that at a flow rate of 7.00 g/min, the composition of the gas is dimethyl ether, methane, carbon dioxide and carbon monoxide. The NIR results further show that at a flow rate of 3.52 g/min, the composition of the gas is dimethyl ether, methane, carbon dioxide and carbon monoxide. There was a much larger amount of carbon dioxide under the slower feed rate (3.52 g/min).

These data show that an alcohol feedstock including methanol can be converted into an ether reaction product using a metal oxide catalyst.

### Example 4

### Synthesis of Diethyl Ether from Ethanol

A reactor system was set up as described in Example 3 above. Etherification was performed using the reactor system with ethanol as a feedstock, instead of methanol.

The reaction conditions for this example are summarized below in Table 3. Notably, the reaction was tested at three different ethanol flow rates, 11.64 g/min, 7.00 g/min, and 3.52 g/min.

TABLE 3

| Reactor | Reactor | Inlet Temp. | Outlet Temp. | | | EtOH | Gases | Burning |
|---|---|---|---|---|---|---|---|---|
| Inlet | Outlet | of heater | of heater | Front | Back | Flow | collection | 1 L gas |
| Temp. | Temp. | exchange | exchanger | Pressure | Pressure | Rate | Rate | time |
| (° C.) | (° C.) | (° C.) | (° C.) | (PSI) | (PSI) | (g/min) | (L/min) | (seconds) |
| 344 | 346 | 357 | 109 | 2500 | 2500 | 3.522 | 0.076 | 132 |
| 345 | 344 | 362 | 111 | 2500 | 2500 | 7.000 | 0.101 | 189 |
| 344 | 346 | 356 | 109 | 2500 | 2500 | 11.64 | No Gases | |

The resulting gas was shown to be combustible by igniting the same and observing it to burn. The mass balance (liquid in versus liquid out) of chemical reactions is shown below in Table 4.

TABLE 4

| | EtOH | EtOH | | |
|---|---|---|---|---|
| EtOH Flow | Flow | Conversion | | EtOH to |
| (g/min) at | (g/min) | Rate | EtOH | Gases |
| Inlet | at Outlet | (g/min) | Recovery | Conversion |
| 3.522 | 3.360 | 0.162 | 95.4% | 4.6% |
| 7.000 | 6.824 | 0.176 | 97.5% | 2.5% |
| 11.64 | 11.62 | 0.020 | 99.8% | 0.2% |

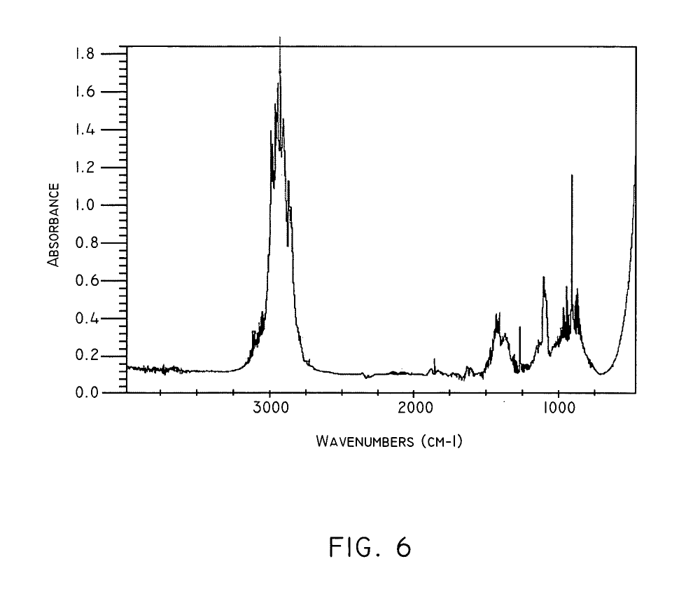

A sample of gas from the reactor was collected in a gas tight cell for NIR (Near Infra-Red) analysis. The NIR spectrum for gas created with an ethanol flow rate of 3.52 g/min is shown in FIG. 6. This was compared with a standard curve NIR spectrum for pure ethane.

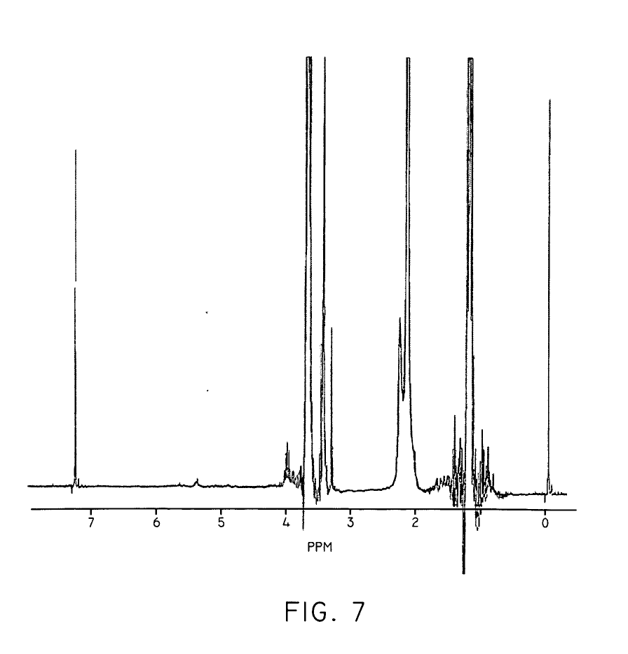

A sample of liquid from the reactor was collected and subjected to NMR (proton nuclear magnetic resonance) analysis. The NMR spectrum for liquid created with an ethanol flow rate of 3.52 g/min is shown in FIG. 7. This was compared with an NMR spectrum for pure diethyl ether.

NIR and NMR results indicated that at a flow rate of 7.00 g/min, the composition of the gas is diethyl ether, methane, and carbon monoxide. The NIR and NMR results further show that at a flow rate of 3.52 g/min, the composition of the gas is diethyl ether, methane, and carbon monoxide. The NIR and NMR results further show that at a flow rate of 11.64 g/min, little gas is produced.

The data show that an alcohol feedstock including ethanol can be converted into an ether reaction product using a metal oxide catalyst.

### Example 5

### Synthesis of Dipropyl Ether from n-Propanol

A reactor system was set up as described in Example 3 above. Etherification was performed using the reactor system with n-propanol as a feedstock, instead of methanol.

The reaction conditions for this example are summarized below in Table 5. Notably, the reaction was tested at two different n-propanol flow rates, 7.00 g/min, and 3.52 g/min.

TABLE 5

| | Reactor | | Inlet Temp. | Outlet Temp. | | | n-PrOH | Gases |
|---|---|---|---|---|---|---|---|---|
| Preheater | Inlet | Reactor | of heater | of heater | Front | Back | Flow | collection |
| Temp. | Temp. | Outlet | exchange | exchanger | Pressure | Pressure | Rate | Rate |
| (° C.) | (° C.) | Temp. (° C.) | (° C.) | (° C.) | (PSI) | (PSI) | (g/min) | (L/min) |
| 350 | 350 | 350 | 225 | 120 | 2500 | 2500 | 3.522 | 0.085 |
| 350 | 350 | 351 | 269 | 151 | 2500 | 2500 | 7.000 | 0.100 |

The resulting gas was shown to be combustible by igniting the same and observing it to burn. The mass balance (liquid in versus liquid out) of chemical reactions is shown below in Table 6.

TABLE 6

| n-PrOH | n-PrOH | | n-PrOH | | |
|---|---|---|---|---|---|
| Flow | Flow | Cold Well | Conversion | | nPrOH to |
| (g/min) | (g/min) at | Collection | Rate | n-PrOH | Gases |
| at Inlet | Outlet | (g/min) | (g/min) | Recovery | Conversion |
| 3.522 | 3.450 | 0.001 | 0.071 | 98.0% | 2.0% |
| 7.000 | 6.917 | 0.005 | 0.078 | 98.9% | 1.1% |

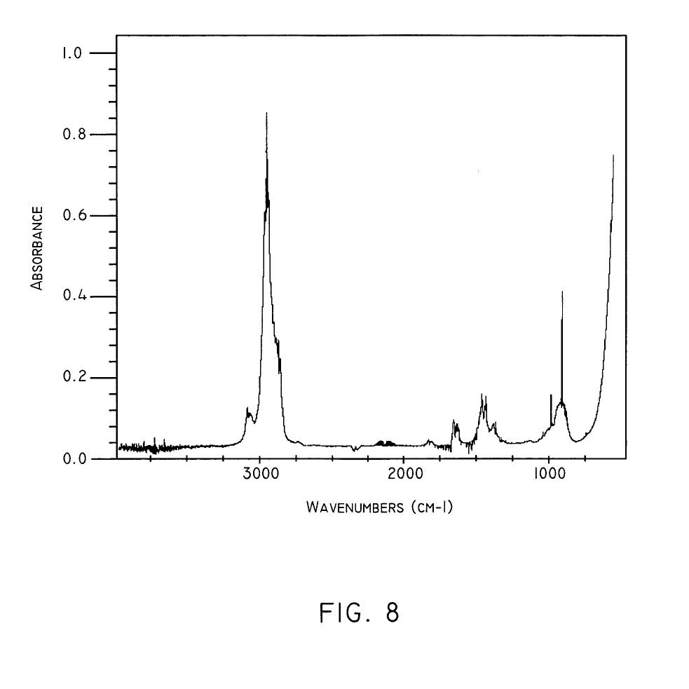

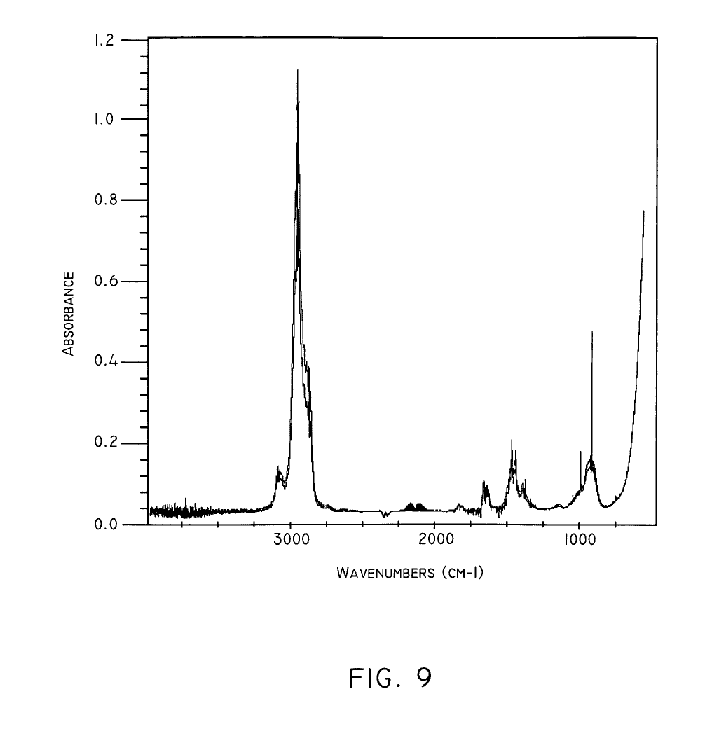

A sample of gas from the reactor was collected in a gas tight cell for NIR (Near Infra-Red) analysis. The NIR spectrum for gas created with an n-propanol flow rate of 3.52 g/min is shown in FIG. 8. The NIR spectrum for gas created with an n-propanol flow rate of 7.00 g/min is shown in FIG. 9. These spectra were compared with standard curve NIR spectra for pure propane and pure propene.

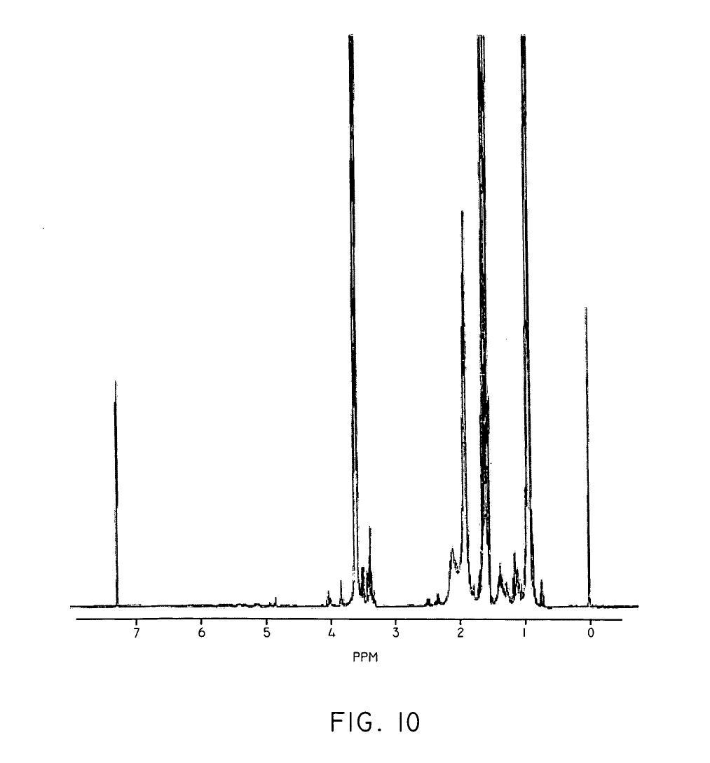

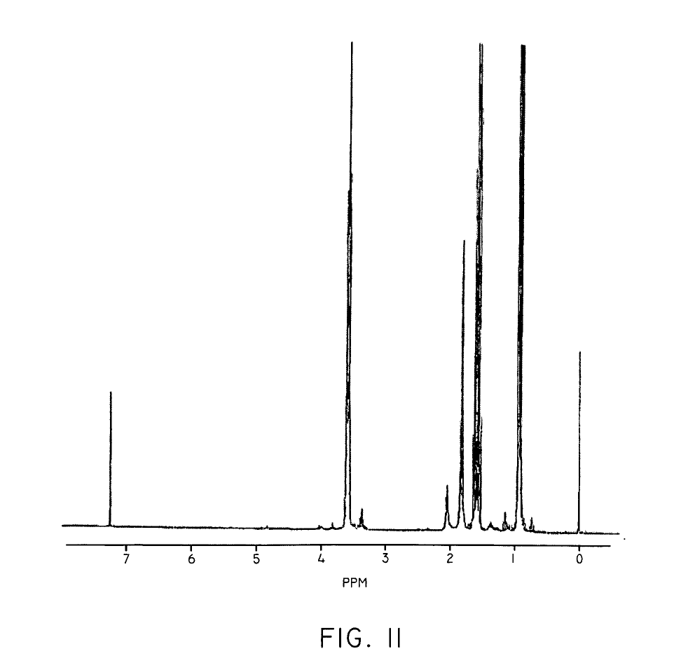

A sample of liquid from the reactor was collected and subjected to NMR (proton nuclear magnetic resonance) analysis. The NMR spectrum for liquid created with an n-propanol flow rate of 3.52 g/min is shown in FIG. 10. The NMR spectrum for liquid created with an n-propanol flow rate of 7.00 g/min is shown in FIG. 11. These were compared with an NMR spectrum for pure n-dipropyl ether.

NIR and NMR results indicated that at a flow rate of 7.00 g/min, the composition of the gas is propene, propane, methane and carbon monoxide. The NIR and NRM results further show that at a flow rate of 3.52 g/min, the composition of the gas is propene, propane, methane, and carbon monoxide. The NIR and NRM results further show that at a flow rate of 11.64 g/min, little gas is produced.

The data show that an alcohol feedstock including n-propanol can be converted into an ether reaction product using a metal oxide catalyst.

### Example 6

### Synthesis of Diethyl Ether (DEE) from Ethanol

A reactor system was set up as described in Example 3 above, with the exception that two separate reactors (both 150×10 mm) packed with bare unmodified titania (80 μm diameter/60 Angstrom average pore diameter) (15.30 grams and 15.24 grams of titania). Etherification was then performed using the reactor system with ethanol as a feedstock. The ethanol feedstock was sparged with pure nitrogen.

The reaction conditions for this example are summarized below in Table 7.

TABLE 7

| | | Inlet | | | | |
|---|---|---|---|---|---|---|
| Reactor | Reactor | Temp. | Outlet Temp. | | | EtOH |
| Inlet | Outlet | of heater | of heater | Front | Back | Flow |
| Temp. | Temp. | exchange | exchanger | Pressure | Pressure | Rate |
| (° C.) | (° C.) | (° C.) | (° C.) | (PSI) | (PSI) | (g/min) |
| 375 | 350 | 221 | 72 | 2350 | 2350 | 0.744 |
| 375 | 352 | 222 | 76 | 2350 | 2350 | 0.744 |
| 375 | 354 | 222 | 81 | 2350 | 2350 | 0.744 |

The resulting gas was shown to be combustible by igniting the same and observing it to burn. The reaction products were identified using NIR and NMR analysis. The average mass balance (liquid in versus liquid out) of chemical reactions is shown below in Table 8. It was determined that 21.1 percent of the ethanol was converted to DEE on a mass in/mass out basis.

TABLE 8

| | EtOH | | Water | |
|---|---|---|---|---|
| EtOH Flow | Flow | DEE | and | |
| (g/min) at | (g/min) | Produced | Organics | Coldwell |
| Inlet | at Outlet | (g/min) | (g/min) | (g/min) |
| 0.744 | 0.48 | 0.16 | 0.11 | 0.0002 |

The data show that an alcohol feedstock including ethanol can be converted into an ether reaction product using a metal oxide catalyst.

The invention has been described with reference to various specific and preferred embodiments and techniques. However, it should be understood that many variations and modifications may be made while remaining within the spirit and scope of the invention.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration to. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

1. A process for synthesizing ethers from an alcohol feedstock comprising:
heating an alcohol feedstock to a temperature greater than about 200 degrees Celsius; and
contacting the alcohol feedstock with a catalyst, the catalyst comprising a metal oxide selected from the group consisting of titania and alumina.

2. The process of claim 1, the metal oxide consisting essentially of titania.

3. The process of claim 1, wherein the operations of heating the alcohol feedstock and contacting the alcohol feedstock with a catalyst are performed simultaneously.

4. The process of claim 1, comprising heating an alcohol feedstock to a temperature greater than about 300 degrees Celsius.

5. The process of claim 1, further comprising subjecting the alcohol feedstock to a pressure greater than about 200 psi.

6. The process of claim 1, the alcohol feedstock comprising a C1-C30 alcohol.

7. The process of claim 1, the alcohol feedstock comprising an alcohol selected from the group consisting of methanol and ethanol.

8. The process of claim 1, the catalyst comprising a particulate metal oxide, the particulate metal oxide comprising an average particle size of about 0.2 microns to about 1 millimeter.

9. The process of claim 1, the catalyst comprising a porous metal oxide having a porosity of about 0.45.

10. The process of claim 1, wherein the metal oxide comprises a Lewis base adsorbed to its surface.

11. A method of synthesizing ethers comprising:
heating an alcohol feedstock to a temperature greater than about 200 degrees Celsius; and
passing the alcohol feedstock through a housing to form a reaction product mixture, the housing comprising a catalyst disposed therein, the catalyst comprising a metal oxide selected from the group consisting of titania and alumina.

12. The method of claim 11, the metal oxide consisting essentially of titania.

13. The method of claim 11, further comprising adding water to the alcohol feedstock.

14. The method of claim 11, comprising heating a alcohol feedstock to a temperature greater than about 300 degrees Celsius.

15. The method of claim 11, further comprising subjecting the alcohol feedstock to a pressure greater than about 200 psi.

16. The method of claim 11, the catalyst comprising a particulate metal oxide, the particulate metal oxide comprising an average particle size of about 0.2 microns to about 1 millimeter.

17. The method of claim 1 1, wherein the operations of heating the alcohol feedstock and passing the alcohol feedstock over a catalyst are performed as part of a continuous process.

18. The method of claim 11, the catalyst comprising a porous metal oxide.

19. The method of claim 18, the porous metal oxide comprising a porosity of between about 0 and 0.8.

20. The method of claim 11, wherein the metal oxide comprises a Lewis base adsorbed to its surface.
